(19) United States
(12) Patent Application Publication
(10) Pub. No.: US 2020/0281783 A1
(43) Pub. Date: Sep. 10, 2020

(54) Sports Training Accessories

(71) Applicant: Musa Al-Sulaimani, State College, PA (US)

(72) Inventor: Musa Al-Sulaimani, State College, PA (US)

(21) Appl. No.: 16/809,002

(22) Filed: Mar. 4, 2020

Related U.S. Application Data
(60) Provisional application No. 62/815,417, filed on Mar. 8, 2019.

(51) Int. Cl. A61F 15/00; A63B 71/14; A63B 71/00; B65H 75/40; B65H 75/44

(52) U.S. Cl. CPC A61F 15/007; A63B 71/14; A63B 71/0036; B65H 75/40; B65H 2701/1924; B65H 75/4402; A63B 2244/102; B65H 2515/805; B65H 75/4486

(57) ABSTRACT

This device disclosed herein can allow portable and efficient rolling and storage of hand wraps, gauze, bandage wraps and ties. In one non-limiting example, the case can be used for athletes in the sport of boxing to neatly roll and store their boxing hand wraps after use. In one non-limiting example, the device can incorporate a manual spindle and reel while another can incorporate a battery and motor to rotate spindle. The case can be incorporated into many different types of materials. Preferred embodiments of this wrap case and roller device and methods of use are disclosed herein.

### CROSS-REFERENCE

This application claims the benefit of U.S. Provisional Application No. 62/815,417, filed on Mar. 8, 2019, which is incorporated herein by reference.

### BACKGROUND OF THE INVENTION

In the world of contact sports, especially boxing, protection of hands from shock during punching is of utmost importance to the sportsperson. Hand wraps are one such accessory used for training by boxers. Hand wraps help to avoid undue pressure and shock on the bones and ligaments of the hand, specifically the knuckles and wrists, by securing them tightly in place and stabilizing them.

Given the importance of hand wraps, it is imperative to take care of them and store them properly so as to prolong their life and to use them effectively. After training, the hand wraps are either rolled up neatly or balled up for storage. Rolling the wraps allows for neat storage and easy next use access but is tedious and time consuming. Balling up the wraps allows for fast storage but wrinkles and tangles the wraps. This problem is not limited to boxing hand wraps. Storing bandage wraps, gauze wraps and ties can be just as difficult. The storage and rolling of these wraps and bandages can be improved through a combined roller/case. There exists a need for a proper case that assists with quick and proper storage of these wraps.

### SUMMARY OF THE INVENTION

The device described herein is a wrap spooling device to store hand wraps, gauze wraps, ties and other similar wraps. It is comprised of a spindle and reel mechanism to effectively store such wraps. In a non-limiting example, it might comprise of an electric spool motor. Some embodiments of the device can also comprise of air holes to air out the wraps. In some embodiments, the spindle may be long enough to accommodate two hand wraps simultaneously. The device can also have a slit guide to properly untangle the wraps before storing them. In some embodiments, the device may have an internal pocket to house a desiccant.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention can be obtained by reference to the following brief description that sets forth illustrative embodiments, in which the principles of the invention are utilized, and the accompanying drawings of which:

FIG. 1 is a photo depicting an exemplary wrap case device in use.

FIG. 2 is a photo of an exemplary device in the closed position.

FIG. 3 is a photo of an exemplary device in an open position revealing the wrap spindle in the middle.

FIG. 4 is a photo depicting a hand wrap being placed in a wrap device in an open configuration in preparation for rolling.

FIG. 5 is a photo depicting the device in the closed position with the wrap being fed through the slit.

FIG. 6 is a photo of the wrap fully rolled and stored inside of a wrap device in an open configuration.

FIG. 7 is a photo from a side perspective view of a wrap device before being closed to store the hand wrap.

FIG. 8 is a side view photo of a device before being closed to store the hand wrap.

FIG. 9 is a photo diagram of a wrap fully stored and stored inside of a wrap device in an open configuration.

### DETAILED DESCRIPTION OF THE INVENTION

The hand wrap case and rolling device disclosed herein can efficiently and effectively roll (i.e. spool) and store a hand wrap, gauze, ties, bandage wraps and any long pliable material (i.e. “wraps”). In one non-limiting example, device can be used by athletes in the sport of boxing to neatly roll and store their hand wraps. In some embodiments, the device can be used to store gauze. In some embodiments, the device can be sued to store bandages. In some embodiments, the device can be used to store wraps. In some embodiments, the device can be used to store ties. One skilled in the art will recognize that rolling device disclosed herein can be used to store any long pliable material.

The device can comprise a mechanism to roll the hand wrap, gauze, ties, bandage wraps or any long pliable material. The rolling mechanism of the device can comprise a spindle and reel mechanism to roll the wrap. In another non-limiting example this device can further comprise an electronic motor attached to the spindle to roll up the wraps.

This device can be made out of any material that will hold the wrap, gauze, ties, bandage wraps, or any long pliable material. The device can comprise a hard material. The device can comprise metal. The device can comprise plastic. The device can comprise leather. The device can comprise rubber. The device can comprise metal. The device can comprise a polymer. The device can comprise polyurethane. Plastic, leather, rubber, metal, and polyurethane are all preferred case materials. Plastic, and metal are preferred spindle materials. Plastic and metal and preferred handle materials. Preferred embodiments of body position training devices and methods of use are disclosed herein.

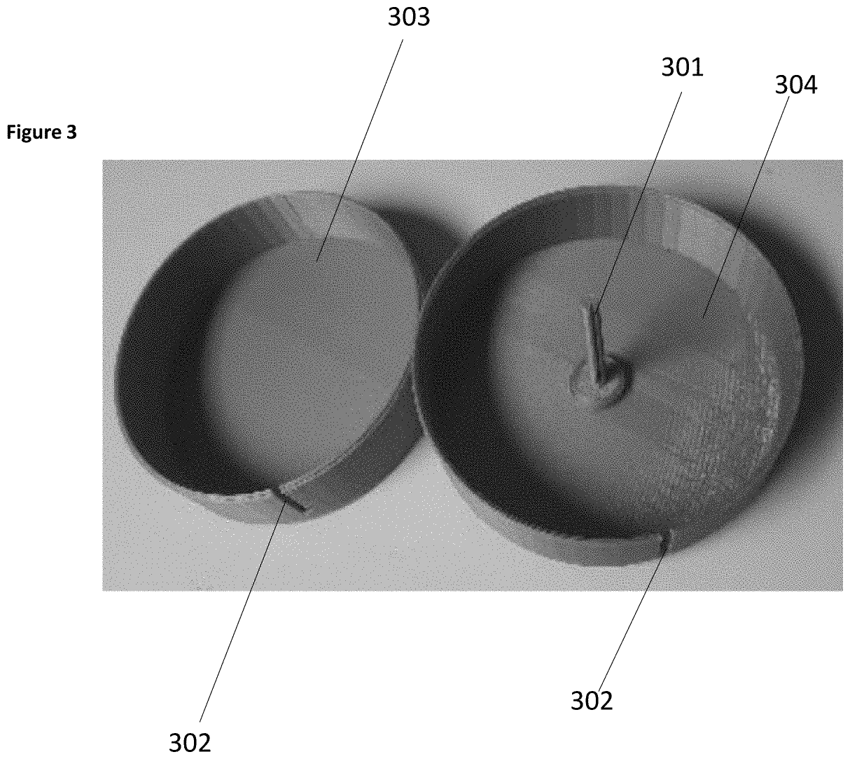

One aspect of the invention described herein comprises a case. The case can be cylindrical in shape. The case can open. The case can be comprised of a first portion and a spindle portion (e.g., see FIG. 3). The case's opening can be comprised of a thread mechanism, a hinge mechanism, a latch mechanism and/or a snap mechanism. The case has a slit and guide for the wrap to enter the case. The case can be attached to a spindle and reel. The spindle is positioned inside of the case to secure and spin the wrap while the reel handle is positioned on the outside of the case for user access. In some embodiments, the reel handle folds into the case for storage and back out for use. In other embodiments a button and motor are used to mechanically reel-in the wraps.

The device disclosed herein can comprise a mechanism for allowing the wrap to be exposed to air. The device disclosed herein can comprise a mechanism for allowing the wrap to dry. The device can comprise air holes, e.g. to air out the wraps. Owing to the high intensity physical nature of contact sports, hand wrap, gauze, ties or bandage wraps can be soaked with sweat. Storing them in such a condition leads to numerous problems such as the material losing elasticity. Air holes in the device can help air out the excessive moisture to keep the hand wrap, gauze, ties or bandage wraps dry for ready use. This device can incorporate any number of air holes to air out the, hand wrap, gauze, ties or bandage wraps stored inside.

In some embodiments, the device can be used with a desiccant to further remove moisture from the wraps. The device disclosed herein can have a small pocket in the interior, e.g., to hold a desiccant. Given the importance of keeping the stored hand wrap, gauze, ties or bandage wraps dry at all times, it might be required to store a desiccant with them. In one non-limiting example, the pocket might be used to store small packets of silica gel.

The device disclosed herein may contain a guide or rail for the wraps to follow. In one non-limiting example, these might be in the form of external protrusions so as to efficiently guide hand wrap, gauze, ties or bandage wraps into the case. This is desirable because knots and tangles in hand wrap, gauze, ties or bandage wraps can cause irreparable damage.

In some embodiments, the spindle can be of a length long to accommodate wrapping two hand wraps side-by-side on a single spindle simultaneously. This is desirable to store multiple wraps at the same time.

An electronic spool motor can be used to power the rotation motion of the spindle. An electronic spool motor may be useful for individuals who have difficulty using a hand-based mechanism (e.g., for a boxer with an injured a hand).

The spool motor can be powered by any power source. In some embodiments a spool is powered by a battery (rechargeable or single use). The spool motor can be powered by an electrical current from a standard wall outlet. The spool motor can be powered by a USB cable connected to a power source.

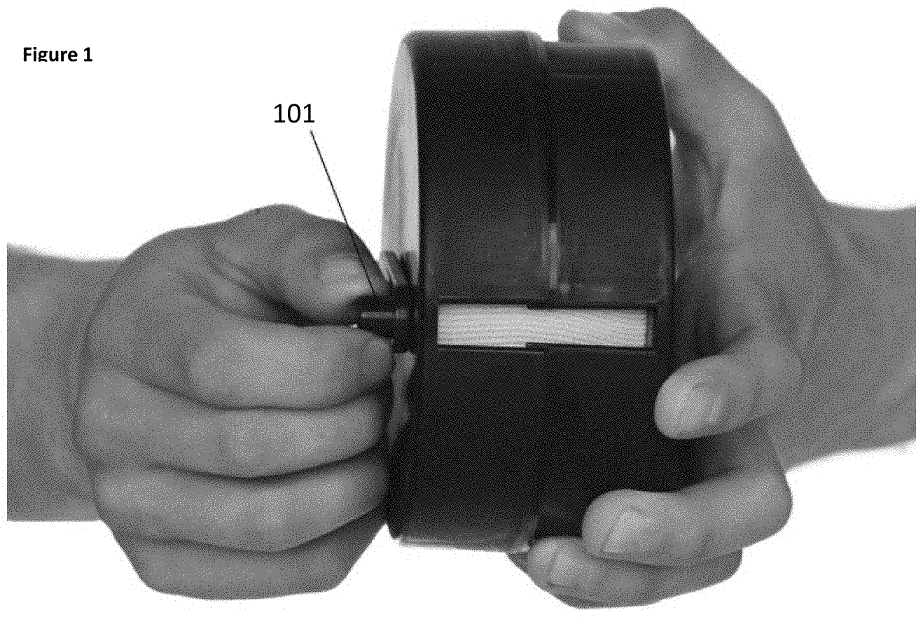

FIG. 1 depicts the device in use. On the outside of the wrap device, a handle is depicted (101). The handle can be connected to the spindle to form a reel that can be used to spool a wrap inside the device. While FIG. 1 depicts the device spooling a wrap in a hand-powered fashion, it is also understood that a motor (e.g., a spool motor) may be connected to the spindle to electronically power the spooling motion.

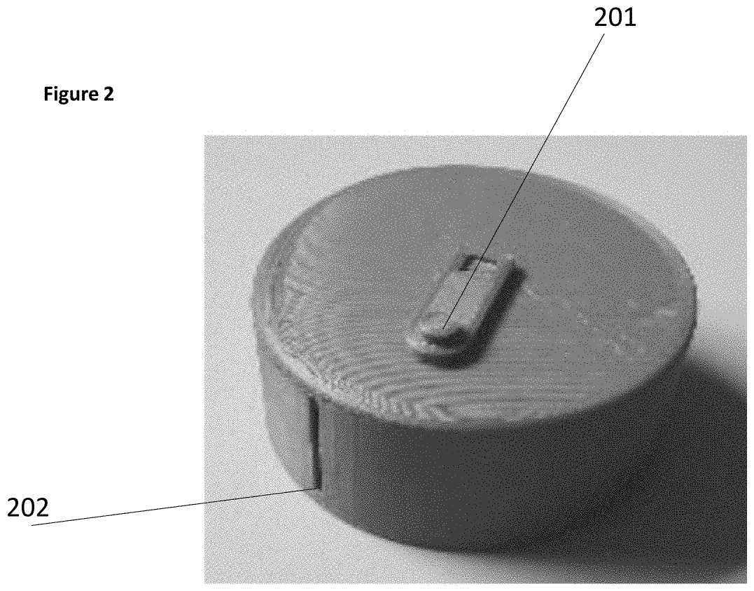

FIG. 2 depicts the device by itself in the closed position. The reel handle (201) is visible on the outside of the device and is connected to the spindle on the inside. The slit opening (202) for the wrap is also depicted in the photo and can be used to feed the wrap into the device while winding the reel handle.

FIG. 3 the device in an open position revealing the wrap spindle (301) where a wrap can be attached (e.g., hooked into a slit down the middle). The spindle can be rotated by a reel which can be located on the exterior. In some embodiments, the spindle rotation may be powered by an electronic spool motor (not shown). The open configuration of the device has a first portion of the case (303) and a spindle portion of the case (304) that can be fitted together. The first portion of the case can further comprise a spindle receiver (not shown) that can accommodate the end of the spindle that is not connected to the spindle portion of the case. The wrap slit (302) can be seen on both the first portion (303) and the spindle portion (304).

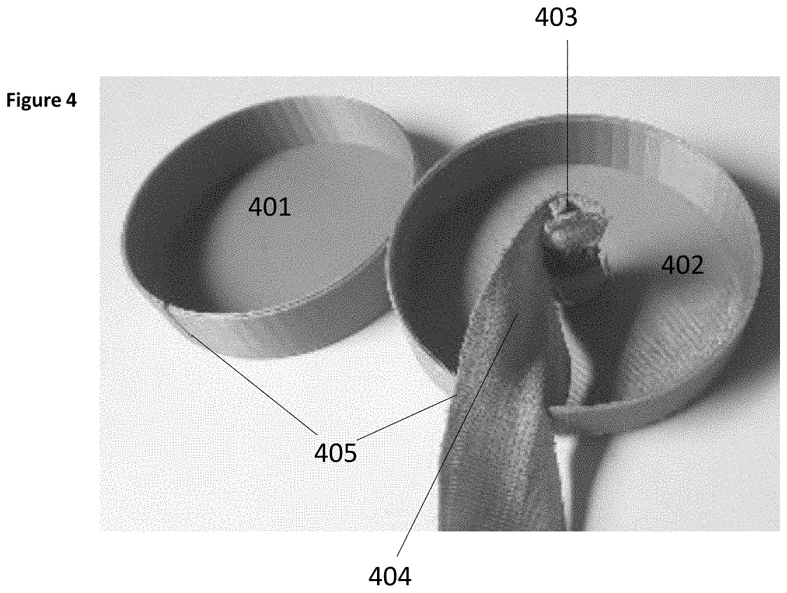

FIG. 4 depicts a hand wrap (404) being placed in the device in preparation for rolling (i.e., spooling). The device is in an open, loading configuration and the first portion of the case (401) is shown along with the spindle portion of the case (402). The visible slit in the spindle (403) allows for insertion of the wrap (404) to begin twisting (spooling). The wrap (404) can be guided into the inside of the device through the device slit (405).

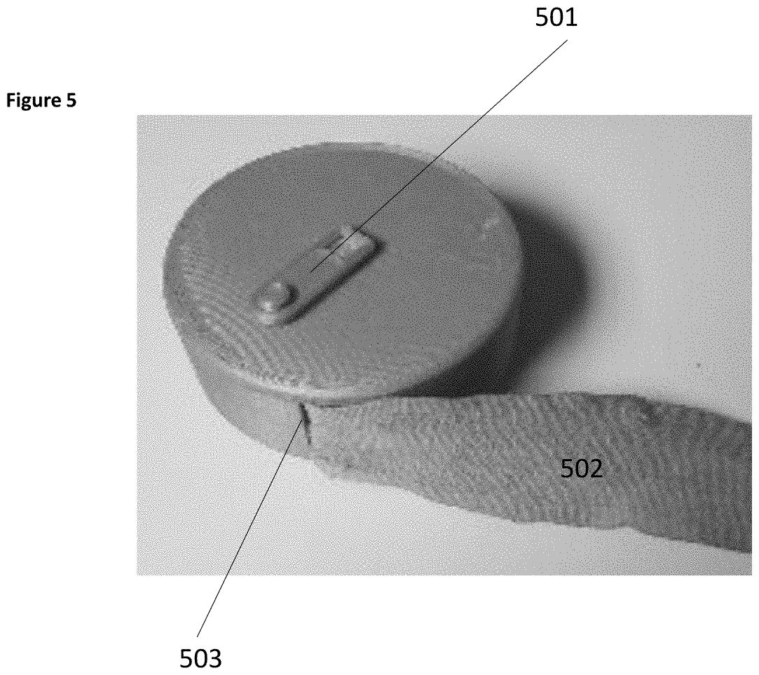

FIG. 5 depicts the device in the closed configuration with the wrap (502) being fed through the slit (503). The handle (501) to power the reel and spindle rotation can be seen.

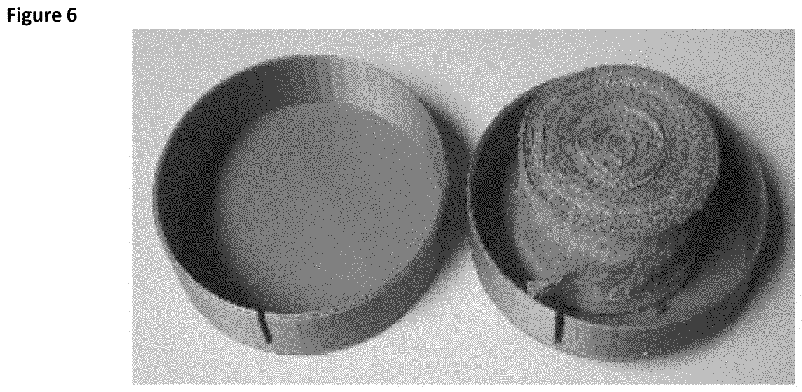

FIG. 6 is a photo of the wrap fully rolled and stored inside of the device. In another non-limiting example, may further comprise a small pocket to house a desiccant, such as silica gel (not shown).

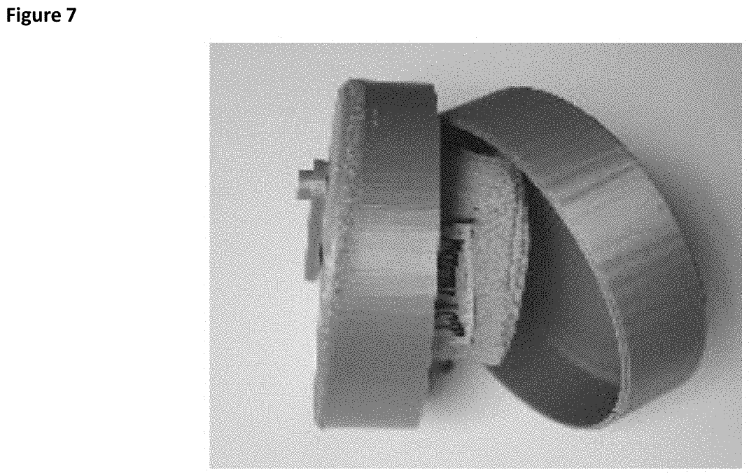

FIG. 7 is a side perspective view of the device before being closed to store the hand wrap.

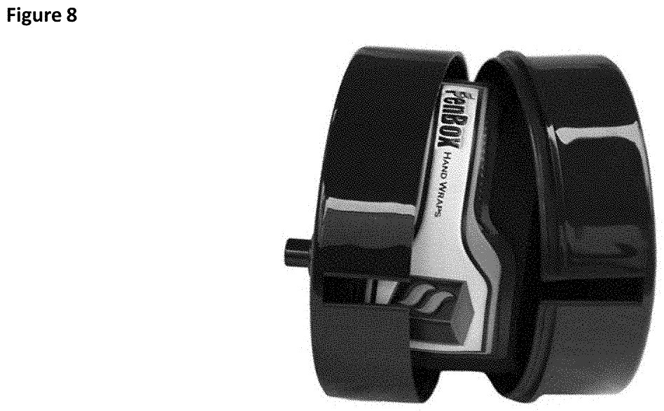

FIG. 8 is an actual photo of the product from the side of the device before being closed to store the hand wrap.

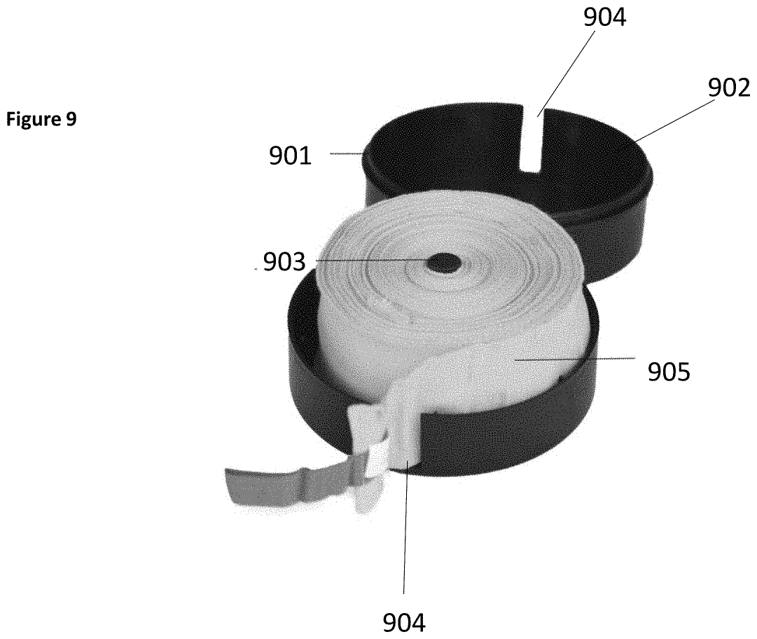

FIG. 9 is a photo of a wrap (905) substantially stored inside of the device. The device comprises a snap lock design (901) that can be used to fit and snap close a first portion (902) of the case with the spindle portion of the case, which each comprise a slit (904) sized to accommodate the wrap (905). The spindle (903) can be seen at the center of the spooled wrap (905).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1) A wrap spooling device, comprising: a body, wherein said body is comprises a case to hold the wraps; and a spindle and reel mechanism wherein said spindle and reel mechanism can be powered to roll a wrap into an interior of said case.

2) The wrap spooling device of claim 1, wherein the device further comprises an electronic spool motor.

3) The wrap spooling device of claim 1, further comprising one or more air holes in said case.

4) The wrap spooling device of claim 3, wherein said one or more air holes comprise 10 or more airholes.

5) The wrap spooling device of claim 1, wherein the spindle is of a length long to accommodate wrapping two hand wraps side-by-side on a single spindle simultaneously.

6) The spooling device of claim 1 further comprising a slit sized to accommodate a wrap.

7) The spooling device of claim 1 further comprising a guide to untangle said hand wrap as it is spooled into the case.

8) The spooling device of claim 1 further comprising a small pocket sized to house a desiccant.
